(19) United States
(12) Patent Application Publication
(10) Pub. No.: US 2025/0281547 A1
(43) Pub. Date: Sep. 11, 2025

(54) Fish Oil Traditional Chinese Medicine Complex with Comprehensive Blood Lipid Regulating Effect and Preparation Method and Application thereof

(71) Applicant: Zhanjiang Bay Laboratory, Zhanjiang (CN)

(72) Inventors: Chun CAI, Zhanjiang (CN); Shuangxi MEI, Zhanjiang (CN); Ao QI, Zhanjiang (CN); Xiaotao CHEN, Zhanjiang (CN); Xike NI, Zhanjiang (CN); Pengfei YAO, Zhanjiang (CN); Kangdi CHEN, Zhanjiang (CN); Danqiao FANG, Zhanjiang (CN)

(73) Assignee: Zhanjiang Bay Laboratory, Zhanjiang (CN)

(21) Appl. No.: 19/073,116

(22) Filed: Mar. 7, 2025

(30) Foreign Application Priority Data
Mar. 7, 2024 (CN) 202410256802.2

(51) Int. Cl. A61K 35/60; A61K 31/202; A61K 36/238; A61K 36/481; A61K 36/65; A61P 3/04; A61P 3/06

(52) U.S. Cl. CPC A61K 35/60; A61K 31/202; A61K 36/238; A61K 36/481; A61K 36/65; A61P 3/04; A61P 3/06

(57) ABSTRACT

Provided is a fish oil-traditional Chinese medicine complex with a comprehensive blood lipid regulating effect, a preparation method, and use thereof. The fish oil-traditional Chinese medicine complex with a comprehensive blood lipid regulating effect includes a fish oil and Astragali Radix, where a volume percentage content of eicosapentaenoic acid (EPA) in the fish oil is 80% or higher. A novel hypolipidemic traditional Chinese medicine complex derived from a deep sea fish oil is developed. The traditional Chinese medicine complex has almost no toxic and side effects, and is safe. A medicament with a multi-target all-round comprehensive blood lipid-regulating function is further provided, which can achieve a combined lipid-lowering effect.

### CROSS REFERENCE TO THE RELATED APPLICATIONS

This application is based upon and claims priority to Chinese Patent Application No. 202410256802.2, filed on Mar. 7, 2024, the entire contents of which are incorporated herein by reference.

### TECHNICAL FIELD

The present disclosure belongs to the technical field of traditional Chinese medicine, and specifically relates to a fish oil-traditional Chinese medicine complex with a comprehensive blood lipid regulating effect and a preparation method and use thereof.

### BACKGROUND

Currently, there are various types of lipid-regulating drugs, which are mainly divided into the following five categories: statins, fibrates, niacins, bile acid sequestrants, and cholesterol absorption inhibitors. However, the treatment with these lipid-regulating drugs has limitations and large side effects. Statins such as lovastatin are mainly to reduce the total cholesterol (TC) and low-density lipoprotein (LDL), and have adverse reactions mainly including liver enzyme elevation, myopathy, and rhabdomyolysis. The liver enzyme elevation is one of the main causes for medication discontinuance. A patient must undergo a liver function test before taking statins. Many patients exhibit poor tolerance to statins. Fibrates such as bezafibrate dispersible tablets are mainly used to lower the triglyceride (TG), and have adverse reactions such as facial flushing, hyperglycemia, hyperuricemia, and upper gastrointestinal discomfort. Niacins such as acipimox capsules are mainly used to increase the high-density lipoprotein (HDL), and have adverse reactions mainly including skin flushing and itching, diarrhea, diminished appetite, possible induction of gastric ulcer in some patients, and blood sugar elevation. Niacins have large side effects, and are generally not used alone as preferred drugs for lipid lowering. Bile acid sequestrants, also known as bile acid resins, include colestipol, which is a common bile acid sequestrant. The common adverse reactions of colestipol are gastrointestinal reactions, nausea, constipation or diarrhea, intestinal obstruction or headache, etc. Cholesterol absorption inhibitors can reduce the blood lipids mainly by inhibiting the absorption of cholesterol in the diet and bile inside the intestinal tract. Currently, there are few cholesterol absorption inhibitors on the market. In summary, after being taken for a long time, the above western medicines lead to obvious side effects, mainly including gastrointestinal symptoms such as nausea, vomiting, and loss of appetite. In severe cases, the long-term administration of the above western medicines can increase the incidence of gallstones. The long-term administration of the above western medicines may also damage the liver and kidneys, trigger the muscle pain, and cause the abnormal blood sugar level in a patient. Currently, there is a lack of drugs with comprehensive blood lipid regulating effect and few side effects. Therefore, it is of great significance to explore and screen traditional Chinese medicine compound prescriptions with comprehensive blood lipid regulating effects.

### SUMMARY

An objective of the present disclosure is to provide a fish oil-traditional Chinese medicine complex with a comprehensive blood lipid regulating effect and a preparation method and use thereof. The present disclosure provides a natural blood lipid-lowering complex that has a remarkable effect, is eco-friendly and safe, and leads to few toxic and side effects.

The present disclosure provides a fish oil-traditional Chinese medicine complex with a comprehensive blood lipid regulating effect, including the following components: a fish oil and Astragali Radix, where a volume percentage content of eicosapentaenoic acid (EPA) in the fish oil is 80% or higher.

Preferably, the fish oil-traditional Chinese medicine complex includes the following components in parts by weight: the fish oil: 10 parts to 30 parts, and Astragali Radix: 100 parts to 200 parts.

Preferably, the volume percentage content of the EPA in the fish oil is 80% or higher.

Preferably, the fish oil-traditional Chinese medicine complex further includes Radix Paeoniae Rubra and Radix Saposhnikoviae.

Preferably, based on a weight of 10 to 30 parts by weight of the fish oil, the the fish oil-traditional Chinese medicine complex includes 20 to 60 parts by weight of Radix Paeoniae Rubra is of and 20 to 60 parts by weight of Radix Saposhnikoviae.

The present disclosure also provides method for preparing the fish oil-traditional Chinese medicine complex described in the above technical solution, including the following steps:
mixing the components other than the fish oil with a solvent, and conducting extraction, filtration, and concentration to produce a concentrate; and
mixing the concentrate with the fish oil to obtain a mixture of the concentrate of the fish oil, concentrating the mixture to produce an extract, and drying the extract to obtain the fish oil-traditional Chinese medicine complex.

Preferably, the solvent includes water.

The present disclosure also provides the use of the fish oil-traditional Chinese medicine complex described in the above technical solution or a fish oil-traditional Chinese medicine complex prepared by the preparation method described in the above technical solution in preparation of a medicament for treating hyperlipidemia.

The present disclosure also provides the use of the fish oil-traditional Chinese medicine complex described in the above technical solution or the fish oil-traditional Chinese medicine complex prepared by the preparation method described in the above technical solution in preparation of a medicament with any one or more effects of the following (1) to (3):
(1) reducing a body weight;
(2) reducing contents of TG, TC, and low-density lipoprotein cholesterol (LDL-C) and increasing a content of high-density lipoprotein cholesterol (HDL-C); and
(3) reducing atherogenic index.

The present disclosure provides a fish oil-traditional Chinese medicine complex with a comprehensive blood lipid regulating effect. The present disclosure combines the inheritance and innovation of traditional Chinese medicine. That is, a fish oil including EPA at a volume percentage content of 80% or higher is combined with Astragali Radix to develop a novel hypolipidemic traditional Chinese medicine complex derived from a deep sea fish oil. The traditional Chinese medicine complex of the present disclosure has almost no toxic and side effects, and is safe. The present disclosure provides a medicament with a multi-target all-round comprehensive blood lipid-regulating function, which can achieve a combined lipid-lowering effect.

### DETAILED DESCRIPTION OF THE EMBODIMENTS

The present disclosure provides a fish oil-traditional Chinese medicine complex with a comprehensive blood lipid regulating effect, including the following components: a fish oil and Astragali Radix. A volume percentage content of EPA in the fish oil is 80% or higher. In the present disclosure, the fish oil-traditional Chinese medicine complex preferably includes the following components in parts by weight: the fish oil: 10 parts to 30 parts, and Astragali Radix: 100 parts to 200 parts. In the present disclosure, the volume percentage content of the EPA in the fish oil is preferably 80% or higher and more preferably 97%. The fish oil-traditional Chinese medicine complex of the present disclosure has a comprehensive blood lipid regulating effect. The blood lipid regulation is a complicated process involving various links such as liver synthesis, intestinal absorption, reverse cholesterol transport, and capture of LDL receptors or scavenger receptors on liver cells. These links do not exist independently, but are interdependent and complementary to each other. Therefore, any monotherapy of a drug with a single action mechanism can only play a limited role. The present disclosure provides a medicament with a multi-target all-round comprehensive blood lipid-regulating function, which can achieve a combined lipid-lowering effect. That is, the medicament can intervene in the synthesis, transport, and metabolism of cholesterol, TG, etc. through multiple pathways and multiple targets, and thus can achieve a great control effect with few adverse reactions. A fish oil, especially a fish oil with a high content (80% or higher) of EPA, has a prominent TG-lowering effect. Astragali Radix includes a specified amount of flavonoids, which can prevent the oxidative accumulation of blood lipids, enhance the activity of antioxidant substances in vivo, and accelerate the metabolism of lipids in the body, thereby achieving a blood lipid-lowering effect. The present disclosure combines a fish oil having an EPA content of 80% or higher with Astragali Radix (Radix Saposhnikoviae and Radix Paeoniae Rubra are further added), which can maximize the blood lipid-regulating effects of the medicinal materials, allows small toxic and side effects, and has a promising clinical medication prospect.

The fish oil-traditional Chinese medicine complex of the present disclosure includes 10 parts to 30 parts, preferably 10 parts to 20 parts, and more preferably 20 parts of fish oil. In the present disclosure, the fish oil is a fish oil with a high EPA content. A volume percentage content of EPA in the fish oil is preferably 80% or higher and more preferably 97%. EPA is an omega-3 fatty acid that can prevent the easy coagulation of blood and reduce a TG level in blood.

The fish oil-traditional Chinese medicine complex of the present disclosure includes 100 parts to 200 parts, preferably 150 parts to 200 parts, and more preferably 200 parts of Astragali Radix. Astragali Radix is a major tonic medicine to invigorate qi and consolidate the surface.

The fish oil-traditional Chinese medicine complex of the present disclosure preferably further includes 20 parts to 60 parts, preferably 25 parts to 60 parts, and more preferably 60 parts of Radix Paeoniae Rubra. Radix Paeoniae Rubra can nourish the blood, replenish yin, and activate the blood to resolve stasis.

The fish oil-traditional Chinese medicine complex of the present disclosure preferably further includes 20 parts to 60 parts, preferably 25 parts to 60 parts, and more preferably 60 parts of Radix Saposhnikoviae. Radix Saposhnikoviae can dispel wind to relieve pain and induce diuresis to alleviate edema.

The present disclosure has no special limitations on the sources of the above raw materials, and the conventional commercially-available products well known to those skilled in the art may be adopted.

The three medicinal materials of Astragali Radix, Radix Paeoniae Rubra, and Radix Saposhnikoviae all can regulate qi and blood and dredge meridians. Astragali Radix has a sweet taste and a slightly warm nature, and belongs to the spleen and lung meridians. Astragali Radix is primarily used to treat the syndrome of spleen qi and lung qi deficiency, the syndrome of spontaneous sweating due to qi deficiency, and the syndrome of qi and blood deficiency. Thus, Astragali Radix is particularly suitable for spleen deficiency and weakened lipid metabolism caused by qi deficiency. Radix Saposhnikoviae has a pungent and sweet taste and a slightly warm nature, and belongs to the bladder, spleen, and liver meridians. Radix Saposhnikoviae has the effect of dispelling wind and relieving the exterior. Radix Paeoniae Rubra has a bitter taste and a slightly cold nature, and belongs to the liver and spleen meridians. Radix Paeoniae Rubra has the effects of clearing heat to cool blood and promoting blood circulation to remove stasis. Since Astragali Radix invigorates qi, Radix Saposhnikoviae facilitates Astragali Radix to replenish qi throughout the body, and Radix Paeoniae Rubra promotes blood circulation to remove meridian obstruction, the three medicinal materials work together to play the effect of tonifying qi and invigorating blood circulation to remove meridian obstruction. Moreover, the omega-3 fatty acid (EPA) in the fish oil possesses a wide range of physiological activities. EPA can effectively inhibit the synthesis of TGs, assist in clearing the fats accumulated on the inner walls of blood vessels, and prevent the further damage of pathogenic factors to the inner walls of blood vessels, thereby inhibiting the deposition of fats on the blood vessel walls. The dyslipidemia is the most closely related to the liver, spleen, and kidneys, and often falls under the categories of “phlegm turbidity,” “blood stasis,” and “qi stagnation.” The combination of the fish oil, Astragali Radix, Radix Saposhnikoviae, and Radix Paeoniae Rubra can not only effectively reduce the contents of TC and TG in terms of biochemical indexes, but also regulate the spleen and kidney deficiency and the liver disorders, thereby playing a comprehensive blood lipid-regulating role throughout the body.

The present disclosure also provides a method of preparing the fish oil-traditional Chinese medicine complex described in the above technical solution, including the following steps:

The components other than the fish oil are mixed with a solvent, and extraction, filtration, and concentration are conducted to produce a concentrate.

The concentrate is mixed with the fish oil, concentration is conducted to produce an extract, and the extract is dried to produce the fish oil-traditional Chinese medicine complex.

In the present disclosure, the components other than the fish oil are mixed with a solvent, and extraction, filtration, and concentration are conducted to produce a concentrate. In the present disclosure, the solvent includes water. In the present disclosure, after the components other than the fish oil are mixed with the solvent, soaking and cooking are preferably conducted. In the present disclosure, the soaking is conducted preferably for 30 min. The present disclosure has no special limitation on methods for the extraction, the filtration, and the concentration, and the conventional extraction, filtration, and concentration methods for traditional Chinese medicine well known to those skilled in the art can be adopted.

In the present disclosure, after the concentrate is produced, the concentrate is mixed with the fish oil, concentration is conducted to produce an extract, and the extract is dried to produce the fish oil-traditional Chinese medicine complex. The present disclosure has no special limitation on methods for the concentration to produce an extract and the drying, and the conventional concentration and drying methods well known to those skilled in the art may be adopted.

The present disclosure also provides the use of the fish oil-traditional Chinese medicine complex described in the above technical solution or a fish oil-traditional Chinese medicine complex prepared by the preparation method described in the above technical solution in preparation of a medicament for treating hyperlipidemia.

The present disclosure also provides the use of the fish oil-traditional Chinese medicine complex described in the above technical solution or a fish oil-traditional Chinese medicine complex prepared by the preparation method described in the above technical solution in preparation of a medicament with any one or more effects of the following (1) to (3):
(1) reducing a body weight;
(2) reducing contents of TG, TC, and LDL-C and increasing a content of HDL-C; and
(3) reducing an atherogenic index.

Explanation of terms:

Hyperlipidemia: Hyperlipidemia, also known as hyperlipoproteinemia, refers to a too-high blood lipid level, and is the most common form of dyslipidemia.

Total cholesterol: Total cholesterol, abbreviated as TC, refers to cholesterol included in various lipoproteins in the serum, namely, a sum of bound cholesterol and free cholesterol.

Triglyceride: Triglyceride, abbreviated as TG, is one of the esters included in the blood. TG is a lipid produced from glycerol and three fatty acids. TG mainly has the function of supplying and storing energy.

Low-density lipoprotein: Low-density lipoprotein, abbreviated as LDL, is a lipoprotein particle that transports cholesterol to peripheral tissue cells.

High-density lipoprotein: High-density lipoprotein, abbreviated as HDL, is a lipoprotein with the highest density and the smallest particle size in the blood. HDL can transport cholesterol from peripheral tissues to various tissue cells, mainly the liver.

Eicosapentaenoic acid: Eicosapentaenoic acid, abbreviated as EPA, is a main component of fish oil. As an omega-3 polyunsaturated fatty acid, EPA is an indispensable important nutrient for human body.

Docosahexaenoic acid: Docosahexaenoic acid, abbreviated as DHA, is a main component of fish oil. As an omega-3 polyunsaturated fatty acid, DHA is an indispensable important nutrient for human body.

In order to further illustrate the present disclosure, the fish oil-traditional Chinese medicine complex with a comprehensive blood lipid regulating effect and the preparation method and application thereof provided by the present disclosure are described in detail below in connection with examples, but these examples should not be understood as limiting the claimed scope of the present disclosure.

It should be noted that the raw materials in the following examples are purchased from the traditional Chinese medicine trading market. A TC assay kit, a TG assay kit, an HDL-C assay kit, and an LDL-C assay kit are purchased from Quanzhou Ruixin Biological Technology Co., Ltd.

SD rats adopted in the pharmacodynamic test are specific pathogen-free (SPF) animals, and are male rats at an age of 2 to 3 months and with a body weight of 180 g to 190 g. SD rats are provided by Guangzhou Yancheng Biotechnology Co., Ltd.

The experimental instruments involved are as follows: Microplate reader: BIO-RAD, USA. Centrifuge: Thermofisher, USA. Vortex mixer: Shanghai Jingke Industrial Co., Ltd. Tianzi balance: Shanghai Jingqi Industrial Co., Ltd. Vacuum drying oven: Shanghai Jingqi Industrial Co., Ltd. Other reagents and instruments not particularly described can be purchased directly.

### Example 1

10 g of fish oil (with an EPA content of 80%), 100 g of Astragali Radix, 20 g of Radix Saposhnikoviae, and 20 g of Radix Paeoniae Rubra were taken. The components other than the fish oil were mixed with water and soaked for 30 min. The soaked medicinal materials were added to water in a volume 5 times the volume of the soaked medicinal materials, heated in a closed environment until the water boiled (the temperature did not exceed 120° C.), and then further heated for 30 min. Filtration was conducted for removing the medicinal residue to produce an extract aqueous solution, and the extract aqueous solution was concentrated to produce a paste. Then the paste was mixed with the fish oil to produce Complex 1.

### Example 2

15 g of fish oil (with an EPA content of 80%), 150 g of Astragali Radix, 25 g of Radix Saposhnikoviae, and 25 g of Radix Paeoniae Rubra were taken. The preparation method was the same as the preparation method in Example 1. Complex 2 was produced.

### Example 3

17 g of fish oil (with an EPA content of 80%), 170 g of Astragali Radix, 40 g of Radix Saposhnikoviae, and 40 g of Radix Paeoniae Rubra were taken. The preparation method was the same as the preparation method in Example 1. Complex 3 was produced.

### Example 4

20 g of fish oil (with an EPA content of 80%), 180 g of Astragali Radix, 40 g of Radix Saposhnikoviae, and 40 g of Radix Paeoniae Rubra were taken. The preparation method was the same as the preparation method in Example 1. Complex 4 was produced.

### Example 5

20 g of fish oil (with an EPA content of 80%), 200 g of Astragali Radix, 60 g of Radix Saposhnikoviae, and 60 g of Radix Paeoniae Rubra were taken. The preparation method was the same as the preparation method in Example 1. Complex 5 was produced.

### Example 6

10 g of fish oil with an EPA content of 80% and 100 g of Astragali Radix were taken. The preparation method was the same as the preparation method in Example 1. Complex 6 was produced.

### Comparative Example 1

A fish oil product with an EPA content of 97% was taken as Control 1.

### Comparative Example 2

10 g of fish oil with an EPA content of 50% and 100 g of Astragali Radix were taken. The preparation method was the same as the preparation method in Example 1. Control 2 was produced.

### Application Example 1

### Sample Preparation

Medicament groups: 40 g of each of Complex 1 to 6 in Examples 1 to 6 was taken and dissolved with 100 mL of water to produce sample solutions. 40 g of each of Controls 1 and 2 in Comparative Examples 1 and 2 was taken and fully dissolved with 100 mL of water to produce control solutions. A 97% EPA-containing fish oil+Astragali Radix group (other conditions were the same as those in Example 6, and only the EPA content in the fish oil was changed) and a 97% EPA-containing fish oil+Astragali Radix+Radix Saposhnikoviae+Radix Paeoniae Rubra group (other conditions were the same as those in Example 1, and only the EPA content in the fish oil was changed) were added. 40 g of each of the complexes produced in these two groups was taken and dissolved with 100 mL of water to produce sample solutions.

### Animal Grouping, Modeling, and Administration

### (2.1) Adaptation Phase

84 SPF male SD rats each with a body weight of (180±20) g were provided by Guangzhou Yancheng Biotechnology Co., Ltd. The experimental rats were raised with an ordinary diet for 7 days. After the experimental rats adapted to the environment, the experiment was started.

### (2.2) Molding Phase and Grouping

The rats were randomly divided into 2 groups based on body weights. 7 rats were fed with an ordinary maintenance diet (Beijing Keao Xieli Feed Co., Ltd., maintenance compound diet for rats and mice) as a normal group. 77 rats were fed with a high-fat diet (irradiated high-fat diet with a fat content of 45%, D12451) as a high-fat group. The body weight was measured once a week. The model group was fed with a model feed for 4 weeks to 6 weeks. Rats in the blank control group and the model group were fasted and subjected to blood collection (the inner canthus). Serum was isolated as soon as possible after blood collection, and serum TC, TG, LDL-C, and HDL-C levels were determined. Compared with the normal group, in the high-fat group, the levels of TC, TG, and LDL-C were significantly increased, and the level of HDL-C was significantly decreased. As a result, it was determined that the rat hyperlipidemia model was established. According to TC levels, the high-fat group was randomly divided into 11 groups (namely, a model group, a 97% EPA-containing fish oil+Astragali Radix traditional Chinese medicine group, a 97% EPA-containing fish oil+Astragali Radix+Radix Saposhnikoviae+Radix Paeoniae Rubra traditional Chinese medicine group, six example groups, and two comparative example groups). After the grouping, there were no significant differences in TC, TG, LDL-C, and HDL-C among the groups except for the normal group.

### (2.3) Test Sample Administration

After the grouping, the administration groups each were orally administered with a test sample at a corresponding dose every day, and the normal group and the model control group each were administered with a same volume of a corresponding solvent. The body weight was measured regularly. The administration cycle was one month. At the end of the experiment, the animals were fasted and subjected to blood collection through the abdominal aorta. Serum was isolated as soon as possible after blood collection, and serum TC, TG, LDL-C, and HDL-C levels were determined.

### Statistical Analysis

Experimental data were processed with the SPSS17.0 statistical software. Experimental results were expressed as x±s. Analysis of variance was conducted, and P<0.05 indicated a significant difference. Statistical results are shown in the table below.

### Statistical Results

(4.1) Body Weight Changes of Rats in Each Group Relative to the Control Group after the Experiment

TABLE 1
Body weight changes of rats in the groups
before and after the experiment (x ± s, g)

| | Before the | After the |
|---|---|---|
| Group | experiment | experiment |
| Normal group | 184 ± 9.6 | 385 ± 9.2 |
| Model group | 185 ± 10.1 | 445 ± 11.4^{▴} |
| 97% EPA-containing fish oil + | 183 ± 9.1 | 427 ± 11.2* |
| Astragali Radix | | |
| 97% EPA-containing fish oil + | 183 ± 8.6 | 419 ± 7.7* |
| Astragali Radix + | | |
| Radix Saposhnikoviae + | | |
| Radix Paeoniae Rubra | | |
| Example 1 | 185 ± 9.3 | 425 ± 10.3* |
| Example 2 | 184 ± 8.7 | 423 ± 9.6* |
| Example 3 | 183 ± 10.3 | 419 ± 9.2* |
| Example 4 | 183 ± 4.9 | 420 ± 8.4* |
| Example 5 | 184 ± 5.2 | 419 ± 7.6* |
| Example 6 | 185 ± 4.8 | 439 ± 9.4* |
| Comparative Example 1 | 184 ± 7.6 | 440 ± 12.7 |
| Comparative Example 2 | 185 ± 9.2 | 442 ± 10.8 |

Notes:
“^{▴}” indicates a significant difference compared with the normal group (P < 0.05), and “*” indicates a significant difference compared with the model control group (P < 0.05).

As shown in Table 1, rats in the model group had a significantly-larger body weight than rats in the normal group. The body weight decreased slightly in the 97% fish oil group (Comparative Example 1), the 50% fish oil group (Comparative Example 2), and the 80% EPA-containing fish oil+Astragali Radix traditional Chinese medicine group (Example 6). The body weight decreased significantly in the 97% EPA-containing fish oil+Astragali Radix group. The average body weight of each of the 80% EPA-containing fish oil+Astragali Radix+Radix Saposhnikoviae+Radix Paeoniae Rubra groups, namely, the five example groups, was significantly lower (P<0.05) than the average body weight of the model group. The body weight of the 97% EPA-containing fish oil+Astragali Radix+Radix Saposhnikoviae+Radix Paeoniae Rubra traditional Chinese medicine group decreased significantly, indicating that the traditional Chinese medicine complex containing a fish oil with an EPA content of higher than 80%, Astragali Radix, Radix Saposhnikoviae, and Radix Paeoniae Rubra had a significant effect of reducing the body weight of a rat.

### (4.2) Changes in Serum Lipid Indexes in Rats of Each Group

Serum TG, TC, LDL-C, and HDL-C in rats were analyzed. High serum TC and TG contents are the main indexes for hyperlipidemia. HDL-C can transport cholesterol in the artery walls to the liver, such that the cholesterol is decomposed into bile acid. HDL-C is an effective anti-arteriosclerosis (AS) factor.

TABLE 2
Comparison of serum indexes among rats of the groups (x ± s)

| | TC | TG | HDL-C | LDL-C |
|---|---|---|---|---|
| Group | mmol/L | mmol/L | mmol/L | mmol/L |
| Normal group | 2.29 ± 0.28 | 0.49 ± 0.05 | 1.43 ± 0.08 | 0.50 ± 0.07 |
| Model group | 2.89 ± 0.17^{▴} | 0.89 ± 0.06^{▴} | 1.07 ± 0.10^{▴} | 0.96 ± 0.25^{▴} |
| 97% EPA-containing fish oil + | 2.69 ± 0.22* | 0.78 ± 0.13* | 1.20 ± 0.36* | 0.78 ± 0.12* |
| Astragali Radix | | | | |
| 97% EPA-containing fish oil + | 2.56 ± 0.21* | 0.77 ± 0.20* | 1.27 ± 0.31* | 0.73 ± 0.14* |
| Astragali Radix + | | | | |
| Radix Saposhnikoviae + | | | | |
| Radix Paeoniae Rubra | | | | |
| Example 1 | 2.62 ± 0.20* | 0.78 ± 0.10* | 1.23 ± 0.08* | 0.75 ± 0.10* |
| Example 2 | 2.61 ± 0.19* | 0.69 ± 0.11* | 1.24 ± 0.09* | 0.74 ± 0.11* |
| Example 3 | 2.59 ± 0.22* | 0.70 ± 0.10* | 1.26 ± 0.10* | 0.74 ± 0.13* |
| Example 4 | 2.56 ± 0.27* | 0.77 ± 0.14* | 1.27 ± 0.09* | 0.73 ± 0.10* |
| Example 5 | 2.55 ± 0.24* | 0.75 ± 0.12* | 1.29 ± 0.07* | 0.73 ± 0.14* |
| Example 6 | 2.76 ± 0.25* | 0.80 ± 0.14 | 1.15 ± 0.20* | 0.80 ± 0.16* |
| Comparative Example 1 | 2.90 ± 0.22 | 0.77 ± 0.15* | 1.06 ± 0.20 | 0.82 ± 0.11 |
| Comparative Example 2 | 2.88 ± 0.21 | 0.82 ± 0.12 | 1.08 ± 0.13 | 0.84 ± 0.13 |

Notes:
“^{▴}” indicates a significant difference compared with the normal group (P < 0.05), and “*” indicates a significant difference compared with the model control group (P < 0.05).

As shown in Table 2, TG, TC, and LDL-C contents in the model group were significantly higher (P<0.05) than TG, TC, and LDL-C contents in the normal group, and the HDL-C content in the model group was significantly lower (P<0.05) than the HDL-C content in the normal group. After the fish oil with an EPA content of 97% (Comparative Example 1) was administered alone, only TG and LDL-C decreased, and the decrease of TG was statistically significant. The 50% EPA-containing fish oil and Astragali Radix prescription (Comparative Example 2) did not have a significant blood lipid-improving effect. After the 80% EPA-containing fish oil+Astragali Radix (Example 6) was administered, TC, TG, and LDL-C decreased significantly, the decreases of TC and LDL-C were statistically significant, and the HDL-C level increased significantly. When Radix Saposhnikoviae and Radix Paeoniae Rubra (Examples 1 to 5) were added on the basis of 80% EPA-containing fish oil+Astragali Radix, the lipid-lowering effect was further enhanced, and the enhanced lipid-lowering effect was comparable to the lipid-lowering effect of the 97% EPA-containing fish oil+Astragali Radix. The combined administration of 97% EPA-containing fish oil, Astragali Radix, Radix Saposhnikoviae, and Radix Paeoniae Rubra allowed the most significant hypolipidemic effect. TG, TC, and LDL-C contents in the 97% EPA-containing fish oil+Astragali Radix+Radix Saposhnikoviae+Radix Paeoniae Rubra group were significantly lower (P<0.05) than TG, TC, and LDL-C contents in the model group. After the combined administration of the 97% EPA-containing fish oil, Astragali Radix, Radix Saposhnikoviae, and Radix Paeoniae Rubra, TC was reduced by about 12%, TG was reduced by about 16%, and LDL-C was reduced by about 23%. The HDL-C content in the 97% EPA-containing fish oil+Astragali Radix+Radix Saposhnikoviae+Radix Paeoniae Rubra group was significantly higher than (P<0.05) the HDL-C content in the model group, and increased by about 20% at most after administration. Results indicate that the combined use of a fish oil with an EPA content of 80% or higher and Astragali Radix allows a significant lipid-lowering effect, and on this basis, the addition of Radix Saposhnikoviae and Radix Paeoniae Rubra leads to an improved hypolipidemic effect. Therefore, in the fish oil-traditional Chinese medicine complex of the present application, the higher the EPA content in the fish oil, the better the lipid-lowering effect.

### (4.3) Changes in Atherogenic Indexes of Rats in Each Group

TABLE 3
Atherogenic indexes of experimental rats in different groups

| Group | AI1 | AI2 |
|---|---|---|
| Normal group | 0.61 ± 0.19 | 0.35 ± 0.05 |
| Model group | 1.70 ± 0.15^{▴} | 0.90 ± 0.06^{▴} |
| 97% EPA-containing fish oil + | 1.24 ± 0.13* | 0.63 ± 0.02* |
| Astragali Radix | | |
| 97% EPA-containing fish oil + | 1.02 ± 0.21 * | 0.57 ± 0.18* |
| Astragali Radix + | | |
| Radix Saposhnikoviae + | | |
| Radix Paeoniae Rubra | | |
| Example 1 | 1.13 ± 0.23* | 0.61 ± 0.03* |
| Example 2 | 1.10 ± 0.21* | 0.60 ± 0.04* |
| Example 3 | 1.06 ± 0.25* | 0.59 ± 0.06* |
| Example 4 | 1.02 ± 0.27* | 0.57 ± 0.05* |
| Example 5 | 0.98 ± 0.26* | 0.57 ± 0.08* |
| Example 6 | 1.40 ± 0.11* | 0.67 ± 0.20* |
| Comparative Example 1 | 1.73 ± 0.12 | 0.75 ± 0.14* |
| Comparative Example 2 | 1.67 ± 0.16 | 0.78 ± 0.19 |

Notes:
“^{▴}” indicates a significant difference compared with the normal group (P < 0.05), and “*” indicates a significant difference compared with the model control group (P < 0.05).

As shown in Table 3, atherogenic indexes AI1 (TC-HDL-C)/HDL-C and AI2 (LDL-C/HDL-C) were significantly increased in rats after high-lipid induction. After the 97% EPA-containing fish oil (Comparative Example 1) was administered alone, only the AI2 index decreased. The 50% EPA-containing fish oil+Astragali Radix prescription (Comparative Example 2) could not reduce the atherogenic indexes. After the 80% EPA-containing fish oil+Astragali Radix (Example 6) was administered, both AI1 and AI2 were reduced. The 97% EPA-containing fish oil+Astragali Radix allowed a better atherogenic index-reducing effect than the 80% EPA-containing fish oil+Astragali Radix. The addition of Radix Saposhnikoviae and Radix Paeoniae Rubra on the basis of the 80% EPA-containing fish oil+Astragali Radix (Examples 1 to 5) allowed an enhanced effect of reducing the atherogenic indexes AI1 and AI2. After the EPA content in the fish oil was further increased (97% EPA-containing fish oil+Astragali Radix+Radix Saposhnikoviae+Radix Paeoniae Rubra), an effect of reducing the atherogenic indexes was improved. Both AI1 and AI2 were significantly reduced compared with the model group (P<0.05), where AI1 was reduced by about 33% and AI2 was reduced by about 26%.

The above test results indicate that the combination of a fish oil with an EPA content of 80% or higher, Astragali Radix, Radix Saposhnikoviae, and Radix Paeoniae Rubra can significantly increase the content of HDL-C and reduce the contents of TC, TG, and LDL-C in the serum of hyperlipidemic rats. The determination results of changes in body weights, contents of TG, TC, HDL-C, and LDL-C, and atherogenic indexes AI1 (TC-HDL-C)/HDL-C and AI2 (LDL-C/HDL-C) of rats before and after the experiment prove that the complex of the present application has a significant hypolipidemic effect.

The fish oil with an EPA content of 80% or higher+Astragali Radix prescription adopted in the present disclosure allows a better lipid-lowering effect than a low-purity fish oil (50% of EPA) prescription. Compared with the administration of a fish oil with an EPA content of 97% alone, the addition of Astragali Radix can improve the lipid-lowering effect. On this basis, the two medicinal materials of Radix Saposhnikoviae and Radix Paeoniae Rubra are added in the present disclosure. It has been found that the 97% EPA-containing fish oil+Astragali Radix+Radix Saposhnikoviae+Radix Paeoniae Rubra has a better lipid-lowering effect than the 97% EPA-containing fish oil+Astragali Radix and the 80% EPA-containing fish oil+Astragali Radix+Radix Saposhnikoviae+Radix Paeoniae Rubra.

In addition, during the entire experiment, the active state of a rat is normal, no physiological lesion is found in each organ during dissection, and the detected biochemical indexes are normal, indicating that the complex of the present application exhibits no obvious influence on the health of tested animals and has high safety.

Although the present disclosure has been described in detail through the above examples, these examples are only some rather than all of the examples of the present disclosure. Other examples may also be acquired by people based on these examples without creative efforts, and all of these examples shall fall within the protection scope of the present disclosure.

What is claimed is:

1. A fish oil-traditional Chinese medicine complex with a comprehensive blood lipid regulating effect, comprising a fish oil and Astragali Radix, wherein a volume percentage content of eicosapentaenoic acid (EPA) in the fish oil is 80% or higher.

2. The fish oil-traditional Chinese medicine complex according to claim 1, comprising the following components in parts by weight: 10 to 30 parts of fish oil, and 100 to 200 parts of Astragali Radix.

3. The fish oil-traditional Chinese medicine complex according to claim 1, further comprising Radix Paeoniae Rubra and Radix Saposhnikoviae.

4. The fish oil-traditional Chinese medicine complex according to claim 3, wherein based on a weight of the 10 to 30 parts of fish oil, the fish oil-traditional Chinese medicine complex comprises 20 to 60 parts by weight of Radix Paeoniae Rubra and 20 to 60 parts by weight of Radix Saposhnikoviae.

5. A method for preparing the fish oil-traditional Chinese medicine complex according to claim 1, comprising the following steps:
mixing components other than the fish oil with a solvent, and conducting extraction, filtration, and concentration to produce a concentrate; and
mixing the concentrate with the fish oil to obtain a mixture of the concentrate and the fish oil, concentrating the mixture to produce an extract, and drying the extract to produce the fish oil-traditional Chinese medicine complex.

6. The method according to claim 5, wherein the solvent comprises water.

7. A method for treating hyperlipidemia, comprising administering the fish oil-traditional Chinese medicine complex according to claim 1 to a subject in need thereof.

8. A method for treating a condition selected from the following items of (1) to (3):
(1) reducing a body weight;
(2) reducing contents of triglyceride (TG), total cholesterol (TC), and low-density lipoprotein cholesterol (LDL-C) and increasing a content of high-density lipoprotein cholesterol (HDL-C); or
(3) reducing an atherogenic index;
comprising administering the fish oil-traditional Chinese medicine complex according to claim 1 to a subject in need thereof.

9. The method according to claim 7, wherein the fish oil-traditional Chinese medicine complex comprises the following components in parts by weight: 10 to 30 parts of fish oil, and 100 to 200 parts of Astragali Radix.

10. The method according to claim 7, wherein the fish oil-traditional Chinese medicine complex further comprises Radix Paeoniae Rubra and Radix Saposhnikoviae.

11. The method according to claim 10, wherein based on a weight of the 10 to 30 parts of fish oil, the fish oil-traditional Chinese medicine complex comprises 20 to 60 parts by weight of Radix Paeoniae Rubra and 20 to 60 parts by weight of Radix Saposhnikoviae.

12. The method according to claim 8, wherein the fish oil-traditional Chinese medicine complex comprises the following components in parts by weight: 10 to 30 parts of fish oil, and 100 to 200 parts of Astragali Radix.

13. The method according to claim 8, wherein the fish oil-traditional Chinese medicine complex further comprises Radix Paeoniae Rubra and Radix Saposhnikoviae.

14. The method according to claim 13, wherein based on a weight of the 10 to 30 parts of fish oil, the fish oil-traditional Chinese medicine complex comprises 20 to 60 parts by weight of Radix Paeoniae Rubra and 20 to 60 parts by weight of Radix Saposhnikoviae.
